(19) United States
(12) Patent Application Publication
(10) Pub. No.: US 2023/0008585 A1
(43) Pub. Date: Jan. 12, 2023

(54) MEDICAL SYSTEMS, DEVICES, AND RELATED METHODS

(71) Applicant: Boston Scientific Medical Device Limited, Galway (IE)

(72) Inventors: Nabarun BHOWMICK, Kolkata (IN); Aditya DHANOTIYA, Indore (IN); Deepak Kumar SHARMA, Muzaffarnagar (IN)

(73) Assignee: Boston Scientific Medical Device Limited, Galway (IE)

(21) Appl. No.: 17/859,044

(22) Filed: Jul. 7, 2022

Related U.S. Application Data
(60) Provisional application No. 63/220,636, filed on Jul. 12, 2021.

(51) Int. Cl. A61B 1/05; A61B 1/00; A61B 1/06

(52) U.S. Cl. CPC A61B 1/053; A61B 1/00105; A61B 1/00066; A61B 1/00128; A61B 1/00124; A61B 1/00101; A61B 1/0676

(57) ABSTRACT

A medical system includes a medical device and an end cap. The medical device includes a handle, including an actuation device, and a shaft extending from the handle. A distal end of the shaft includes a tab, and the tab is movable based on movement of the actuation device. The end cap includes a modular camera assembly. The modular camera assembly includes a camera, a support, an arm, and a cavity. The tab is coupleable to the modular camera assembly via the cavity, and movement of the tab controls a position of the support and the camera.

### CROSS-REFERENCE TO RELATED APPLICATIONS

This application claims the benefit of priority under 35 U.S.C. § 119 from U.S. Provisional Application No. 63/220,636, filed on Jul. 12, 2021, which is incorporated by reference herein in its entirety

### TECHNICAL FIELD

Various aspects of this disclosure generally relate to modular and/or reusable medical systems, devices, and related methods. For example, the disclosure includes systems, devices, and related methods for utilizing one or more medical systems or devices including reusable and disposable components to visualize, manipulate, or otherwise treat a target site of a patient.

### BACKGROUND

Various medical systems and devices may be utilized in numerous procedures for treating multiple patients. Prior to reuse, such medical systems or devices may undergo extensive sterilization and/or reprocessing procedures to safely prepare the system or device for use in a subsequent procedure. However, despite extensive cleaning measures, cross-contamination between patients may still occur from the reuse of medical systems or devices across multiple procedures, thereby resulting in possible infection and other post-procedure complications for the patient. Disposable medical systems or devices may be employed in lieu of reusable medical systems devices. However, providing for a single use of components may result in increased costs, materials, etc. These concerns may increase the duration, costs, and risks of the medical procedure. The systems, devices, and methods of this disclosure may rectify some of the deficiencies described above or address other aspects of the art.

### SUMMARY

Examples of this disclosure relate to, among other things, systems, devices, and methods for performing one or more medical procedures. Each of the examples disclosed herein may include one or more of the features described in connection with any of the other disclosed examples.

In one example, a medical system may include a medical device and an end cap. The medical device may include a handle, which may include an actuation device, and a shaft extending from the handle. A distal end of the shaft may include a tab, and the tab may be movable based on movement of the actuation device. The end cap may include a modular camera assembly. The modular camera assembly may include a camera, a support, an arm, and a cavity. The tab may be coupleable to the modular camera assembly via the cavity, and movement of the tab may control a position of the support and the camera.

The medical system may include one or more of the following features. The actuation device may be physically and electrically connected to the tab via an actuation wire. The modular camera assembly may include one or more mating elements. The tab may include one or more mating elements on a distal end of the tab. The one or more mating elements on the modular camera assembly and the one or more mating elements on the tab may be configured to electrically connect the handle to the camera. The arm may further include one or more connection wires that electrically connect the camera to the one or more mating elements on the modular camera assembly.

The distal end of the shaft may include a peg, and the end cap may include a slot configured to receive the peg. The slot may include a longitudinal portion and a partially circumferential portion extending from the longitudinal portion. The modular camera assembly and a body portion of the end cap may form a passage that connects to the cavity. The longitudinal portion of the slot may be spaced away from the passage along an interior circumference of the end cap. The handle may include a control device. The distal end of the shaft may include a flap. Movement of the control device may be configured to adjust a position of the flap between at least a first configuration and a second configuration. When the end cap is coupled to the distal end of the shaft, the flap may at least partially align with the modular camera assembly.

The modular camera assembly may include a spring assembly configured to bias the tab distally. The end cap may further include a body portion and a cover portion, and the support may be pivotably coupled the body portion of the end cap. The body portion, the cover portion, and the modular camera assembly may be separable and coupleable. The handle may include a port, and the port may be in communication with a distal opening in the distal end of the shaft. The end cap may include a proximal portion and a distal portion. The proximal portion may be substantially cylindrical, and the distal portion may be substantially semi-cylindrical. The end cap may further include one or more illumination devices positioned on the support.

In another aspect, an end cap for a medical device may include a body portion, a cover portion, and a modular camera assembly. The modular camera assembly may include a camera, a support, an arm, and a cavity. The camera may be coupled to the support. Movement of the arm may be configured to rotate the support and adjust a field of view of the camera.

The end cap for the medical device may include one or more of the following features. The modular camera assembly may further include one or more mating elements. The arm may further include one or more connection wires that electrically connect the camera to the one or more mating elements. The end cap may further include an illumination device coupled to the support. The one or more connection wires may electrically connect the illumination device to the one or more mating elements.

In yet another aspect, a method may include coupling an end cap to a distal end of a shaft of a first medical device. The end cap may include a modular camera assembly. The modular camera assembly may include a camera, a support, an arm, and a cavity. The first medical device may include a handle and a shaft. The handle may include an actuation device. A distal end of the shaft may include a tab. The tab may be physically coupled to the actuation device via an actuation wire. The end cap and the shaft may be coupled by at least positioning the tab within the cavity. The method may further include performing a first medical treatment. Performing the medical treatment may include either directing a treatment device with an elevator of the first medical device or directing a field of view of the camera. The method may further include uncoupling the end cap from the distal end of the shaft of the first medical device, cleaning the end cap, coupling the end cap to a second medical device, and performing a second medical treatment.

The method may include one or more of the following features. The end cap may further include a body portion and a cover portion. The step of cleaning the end cap may include separating the body portion, the cover portion, and the modular camera assembly.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 illustrates a perspective view of an exemplary medical system, including a medical device and an end cap, with a distal portion of the medical system enlarged, according to aspects of this disclosure.

FIG. 2A is a perspective view of a distal end of the medical device and the end cap in an uncoupled configuration, and FIG. 2B is a side view of the distal end of the medical device and the end cap in the uncoupled configuration, according to aspects of this disclosure.

FIGS. 3A-3C illustrate side views of various portions of the end cap, according to aspects of this disclosure.

FIGS. 4A-4D illustrate various views of the medical device and the end cap in the uncoupled configuration or in a coupled configuration, according to aspects of this disclosure.

FIGS. 5A-5F illustrate various views of the end cap in first and second configurations, according to aspects of this disclosure.

FIG. 6 is a flow diagram of an exemplary method, according to aspects of this disclosure.

### DETAILED DESCRIPTION

The terms “proximal” and “distal” are used herein to refer to the relative positions of the components of an exemplary medical system and exemplary medical devices. When used herein, “proximal” refers to a position relatively closer to the exterior of the body or closer to a medical professional using the medical system or medical device. In contrast, “distal” refers to a position relatively further away from the medical professional using the medical system or medical device, or closer to the interior of the body. As used herein, the terms “comprises,” “comprising,” “having,” “including,” or other variations thereof, are intended to cover a non-exclusive inclusion, such that a system, device, or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value.

Examples of this disclosure include systems, devices, and methods for facilitating and/or improving the efficacy, efficiency, cost, and/or safety of a medical procedure. Embodiments of the disclosure may relate to systems, devices, and methods for performing various medical procedures and/or treating portions of the large intestine (colon), small intestine, cecum, esophagus, stomach, any other portion of the gastrointestinal tract, and/or any other suitable patient anatomy (collectively referred to herein as a “treatment site”). Various embodiments described herein include single-use or disposable medical devices. Some aspects of the disclosure may be used in performing an endoscopic, arthroscopic, bronchoscopic, ureteroscopic, colonoscopic, or other type of procedure. For example, the disclosed aspects may be used as or with duodenoscopes, bronchoscopes, ureteroscopes, colonoscopes, catheters, diagnostic or therapeutic tools or devices, or other types of medical devices. One or more of the elements discussed herein could be metallic, plastic, or include a shape memory metal (such as nitinol), a shape memory polymer, a polymer, or any combination of biocompatible materials.

Reference will now be made in detail to examples of the disclosure described above and illustrated in the accompanying drawings. Wherever possible, the same reference numbers will be used throughout the drawings to refer to the same or like parts. It is noted that one or more aspects of the medical systems, devices, and methods discussed herein may be combined and/or used with one or more aspects of other medical systems or devices discussed herein.

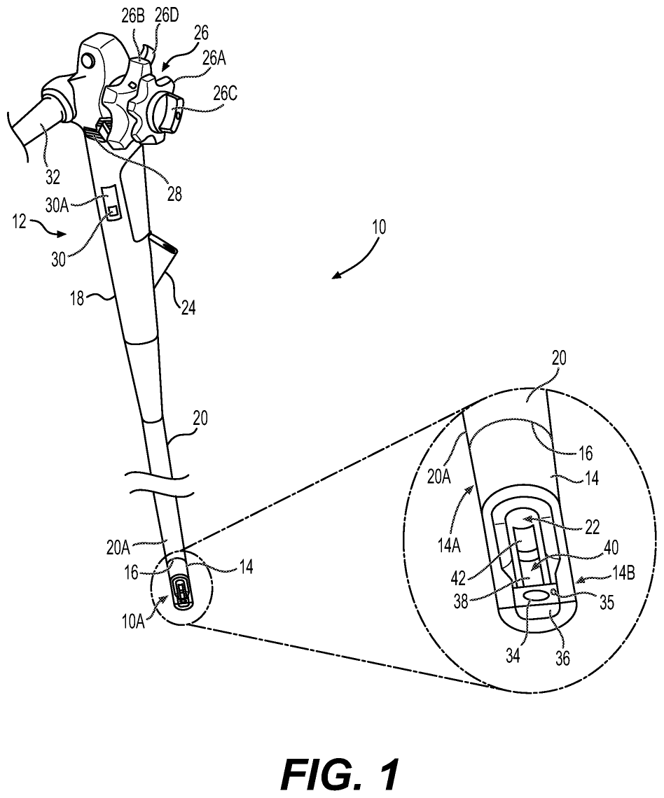

FIG. 1 illustrates a perspective view of an exemplary medical device system 10 that includes a medical device 12 and an end cap 14. Respective portions of medical device 12 and end cap 14 may be coupled to form a coupling 16. Medical device 12 and end cap 14 may be selectively coupled to form medical device system 10, including a distal portion 10A. As discussed below, medical device 12 may be disposable, and one or more portions of end cap 14 may be reusable. Additionally, coupling 16 may help to allow for control of and/or actuation of one or more aspects of end cap 14 via one or more controls and/or actuators on medical device 12.

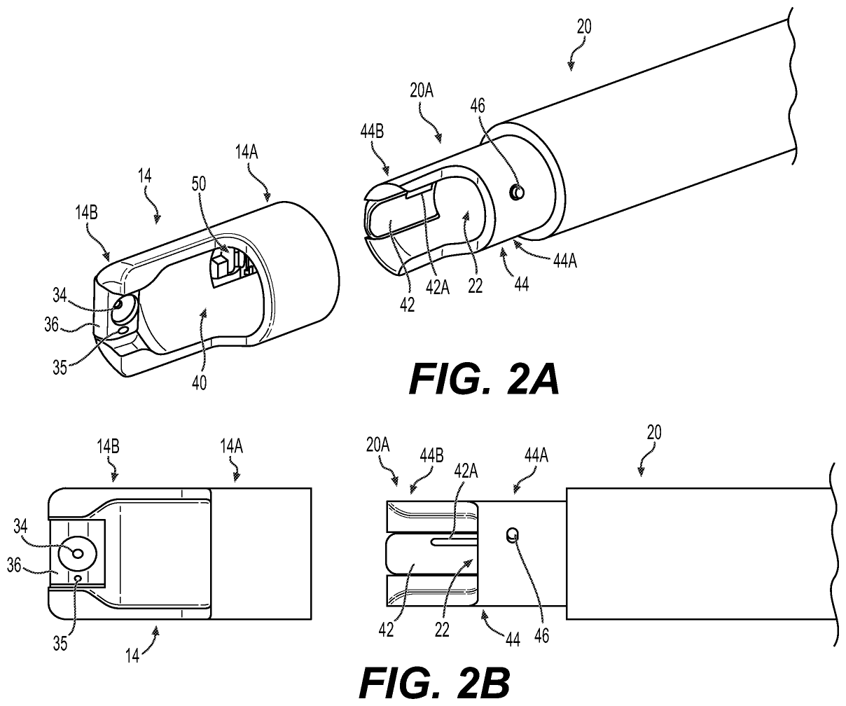

Medical device 12 includes a handle 18, including a handle body, and a shaft 20, for example, extending from a distal end of handle 18 to a distal end 20A. As shown in FIGS. 2A and 2B, medical device 12 also includes at least one working channel with a distal opening 22. For example, the working channel may extend from handle 18 to distal opening 22 in distal end 20A. Medical device 12 also includes a port 24, for example, on handle 18, and port 24 may connect to the working channel such that the working channel connects port 24 to distal opening 22. A tool or other accessory device may be inserted through port 24 and the working channel, to distal opening 22.

Medical device 12 may include a first control device 26, for example, on a portion of handle 18 (e.g., a proximal portion of handle 18), and one or more portions of first control device 26 may be manipulated (e.g., rotated, actuated, etc.) to control a movement of one or more portions of distal end 20A. Medical device 12 may also include a second control device 28, for example, on a portion of handle 18 (e.g., an intermediate portion of handle 18). In one aspect, second control device 28 may be a lever, button, knob, or slider movable relative to handle 18, for example, to control another movement of one or more portions of distal end 20A. As discussed below, second control device 28 may control the movement of an elevator, for example, to direct a treatment device delivered through the working channel and out of distal opening 22, for example, to treat the treatment site. Although not shown, medical device 12 may include a locking mechanism to secure a position of second control device 28 relative to handle 18.

Additionally, medical device 12 may include an actuation device 30, for example, movable within an actuation device slot 30A on another portion of handle 18 (e.g., an intermediate portion of handle 18), to control another movement of one or more portions of distal end 20A. Although not shown, medical device 12 may include a locking mechanism to secure a position of actuation device 30 (such as a knob or a slider) within actuation device slot 30A. As discussed in greater detail below, end cap 14 may be coupled to distal end 20A, and movement of actuation device 30 within actuation device slot 30A may control one or more aspects of end cap 14.

Medical device 12 may be a duodenoscope, an endoscope, a colonoscope, an ureteroscope, a bronchoscope, etc., or any other like device having a handle and a shaft. As mentioned, medical device 12 may include first control device 26, for example, on a proximal portion of handle 18. First control device 26 may be movable (e.g., rotatable) relative to a remainder of handle 18, and may control the movement of a portion (e.g., distal end 20A) of shaft 20. First control device 26 may include one or more dials or knobs. As shown in FIG. 1, first control device 26 may include first and second knobs 26A and 26B, for example, each rotatable to deflect distal end 20A along two different planes. In one or more aspects, first knob 26A may deflect distal end 20A along a first plane (e.g., up or down), and second knob 26B may deflect distal end 20A along a second plane (e.g., left or right) that is perpendicular to the first plane. First control device 26 may include one or more locking mechanisms. As shown in FIG. 1, first control device 26 may include one or more locking mechanisms 26C, 26D, for example, engageable with one or more of knobs 26A and 26B to lock and/or unlock the position of the knob, and thus lock and/or unlock the position of distal end 20A of shaft 20. For example, locking mechanism 26C may be engageable with one or more or both of knobs 26A and 26B, and locking mechanism 26D may be engageable with the other one or both of knobs 26A and 26B. Furthermore, in another aspect, second control device 28 may actuate or move one or more elevators (e.g., flap 42), or otherwise actuate a cable driven function of medical device 12.

Medical device 12 may include a conduit 32, often called an umbilicus. For example, handle 18 may be coupled to conduit 32. Conduit 32 may connect handle 18 to an external power source, processing software, one or more displays, one or more memory or storage devices, etc. In one or more aspects and as discussed below, end cap 14 may include one or more illumination devices and/or cameras, which may be powered and/or connected to processing software, one or more displays, a memory, etc. via one or more communication wires (not shown) within end cap 14, medical device 12, and conduit 32. Alternatively or additionally, medical device 12 and/or end cap 14 may include one or more internal energy sources (e.g., an internal battery in handle 18). Furthermore, conduit 32 may connect handle 18 to one or more fluid sources, for example, an air source, a water source, etc. Conduit 32 may also connect handle 18 to a suction source. In these aspects, one or more valves (not shown) coupled to and/or received within one or more apertures (not shown) in handle 18 may control the delivery of air or water and/or the application of suction through medical device 12 to a treatment site distal to distal end 20A, for example, distal to end cap 14.

As shown in the enlarged portion of FIG. 1, end cap 14 may include a proximal portion 14A and a distal portion 14B. Proximal portion 14A may be coupled to distal end 20A of shaft 20, and may be generally cylindrical. Distal portion 14B may be generally semi-cylindrical. Furthermore, distal portion 14B may include at least one camera 34 (an imaging device). Distal portion 14B may also include at least one illumination device 35. As discussed in detail below, camera 34 and/or illumination device 35 may be positioned on a support 36. Support 36 may be movable, for example, pivotable. Support 36 may be coupled to an arm 38, and movement of arm 38 may control the movement of support 36. In one aspect and as discussed in detail below, movement of actuation device 30 within actuation device slot 30A may control the movement of arm 38, and thus control the movement of support 36. Movement of support 36 may change the field of view of camera 34 and/or the field of illumination of illumination device 35. Furthermore, end cap 14 may include an end cap channel 40, for example, that connects to distal opening 22 of the working channel of medical device 12 when end cap 14 is coupled to distal end 20A of shaft 20. As shown in FIG. 2A, end cap channel 40 may be generally cylindrical within proximal portion 14A of end cap 14, and channel 40 may be generally semi-cylindrical within distal portion 14B of end cap 14.

Moreover, as shown in the enlarged portion of FIG. 1, with end cap 14 coupled to distal end 20A of shaft 20, a flap 42 (an elevator) may be positioned within a portion of end cap 14. As discussed below, flap 42 may extend from a portion of distal end 20A. Moreover, flap 42 may be moveable, for example, via movement of second control device 28. In this aspect, flap 42 may be movable between at least a first configuration and a second configuration, and movement between the first and second configurations may help to direct a treatment device delivered through the working channel and out of distal opening 22, for example, to treat the treatment site.

FIG. 2A is a perspective view of end cap 14 separated from distal end 20A of shaft 20, and FIG. 2B is a side view of end cap 14 separated from distal end 20A of shaft 20. As shown, end cap 14 includes camera 34, illumination device 35, and support 36. Moreover, distal end 20A may include a projection 44, which may be configured to be received within a portion of end cap 14. For example, projection 44 may include a proximal portion 44A and a distal portion 44B. Proximal portion 44A of projection 44 may be substantially cylindrical, and distal portion 44B of projection 44 may be substantially semi-cylindrical, for example, corresponding to the sizes and/or shapes of proximal portion 14A and distal portion 14B of end cap 14.

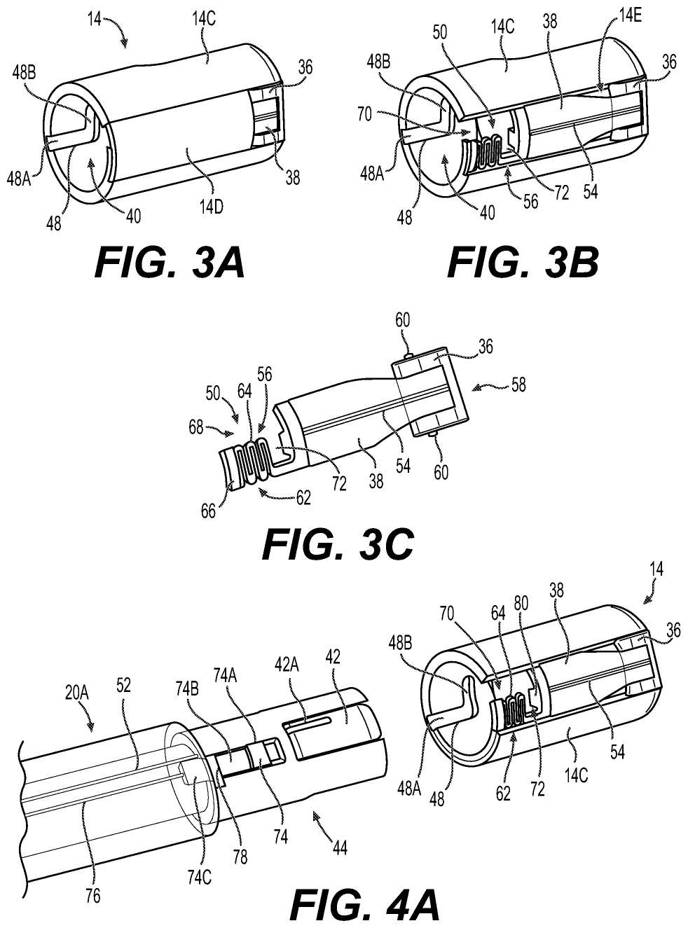

In one aspect, proximal portion 44A may include one or more pegs 46, for example, extending radially outward. As shown in FIGS. 3A and 3B, end cap 14 may include one or more slots 48, and peg 46 may be received within one or more slots 48, for example, to help couple distal end 20A to end cap 14. As shown in FIGS. 3A and 3B, slot 48 may include a longitudinal portion 48A and a partially circumferential portion 48B. Peg 46 may be inserted into longitudinal portion 48A, for example, and advanced longitudinally within slot 48. Then, with peg 46 aligned with partially circumferential portion 48B, rotating peg 46 may advance peg 46 within partially circumferential portion 48B, for example, to help secure and/or otherwise couple distal end 20A to end cap 14.

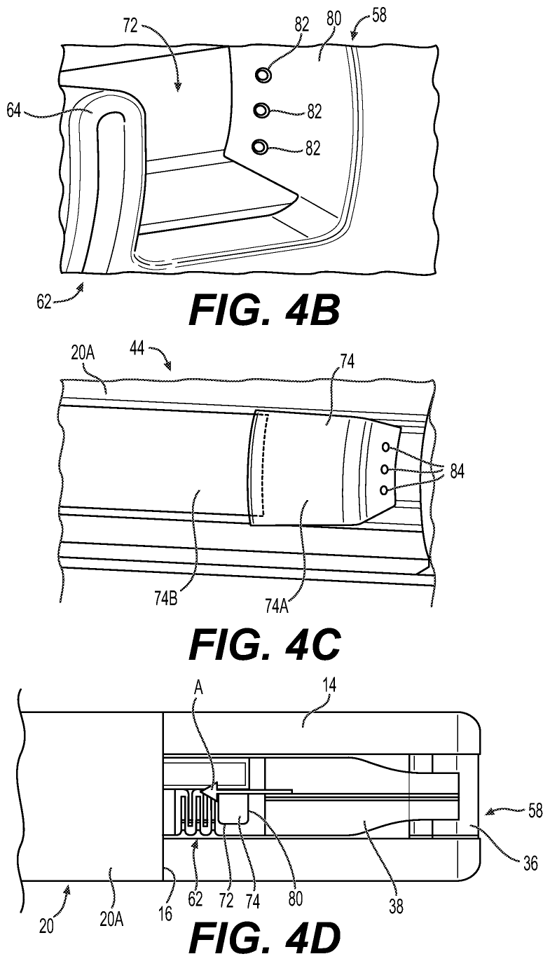

As mentioned above, distal end 20A may include flap 42. Flap 42 may extend distally from proximal portion 44A, for example, parallel to distal portion 44B. As discussed below, end cap 14 may include an interface portion 50, for example, on a portion of proximal portion 14A. Interface portion 50 may be coupled to arm 38, as shown in FIGS. 3B, 3C, and 4A. When end cap 14 is coupled to distal end 20A, flap 42 may at least partially overlap with interface portion 50.

Moreover, flap 42 may include a flap lumen, groove, or opening 42A. Opening 42A may receive one or more control wires 52 (FIG. 4A). For example, the one or more control wires 52 may be coupled to one or more portions of second control device 28, and movement of second control device 28 may move (e.g., extend or retract) control wire(s) 52, for example, to manipulate flap 42. In one aspect, movement of second control device 28 may move control wire(s) 52 in order to manipulate flap 42 from the straight configuration shown in FIGS. 2A and 2B to an angled configuration (not shown), for example, such that flap 42 serves as an elevator to direct one or more treatment devices delivered through distal opening 22 of working channel to a side of distal end 20A. For example, flap 42 may direct one or more treatment devices through the semi-cylindrical opening formed by distal portion 44B of projection 44 and distal portion 14B of end cap 14. Furthermore, flap 42 may be manipulated through a range of positions to direct the one or more treatment devices relative to distal portion 10A of medical system 10 and/or the treatment site. For example, flap 42 may be positioned in the straight configuration (FIGS. 2A and 2B), and manipulation of second control device 28 may control flap 42 from the straight configuration to one or more angled positions, for example, to direct the one or more treatment devices delivered through the working channel and out of distal opening 22 toward the side of distal end 20A.

FIGS. 3A-3C illustrate perspective views of various portions of the end cap 14. As mentioned, and as shown in FIGS. 3A and 3B, a body portion 14C of end cap 14 may include slot 48, with longitudinal portion 48A and partially circumferential portion 48B, for example, to receive peg 46 (FIGS. 2A and 2B) to couple distal end 20A of shaft 20 to end cap 14. Moreover, end cap 14 may include a lid or cover portion 14D, for example, spanning and/or coupled to a portion of body portion 14C. In one or more aspects, cover portion 14D may at least partially overlap with flap 42. For example, cover portion 14D may be positioned approximately opposite to slot 48, for example, on opposing sides of end cap channel 40.

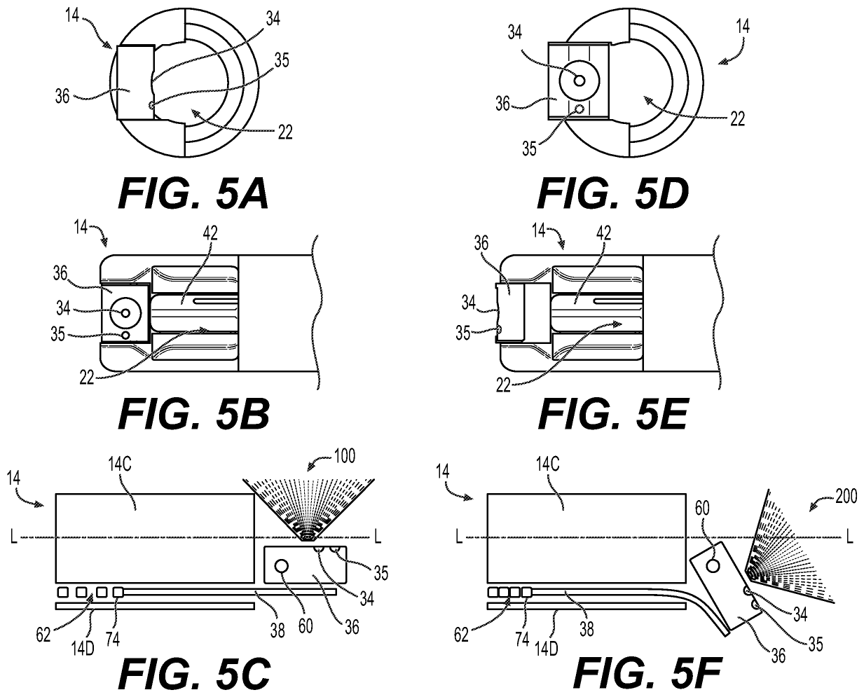

FIG. 3B illustrates end cap 14 with cover portion 14D removed. As shown, end cap 14 includes support 36, arm 38, and end cap channel 40. Arm 38 may be positioned with an aperture 14E, for example, an aperture extending longitudinally through body portion 14C. In this aspect, arm 38 may be moveable within aperture 14E, for example, to pivot support 36, and thus also pivot camera 34 and illumination device 35 (FIGS. 2A, 2B, and 5A-5F). Additionally, one or more connection wires 54 may be positioned on and/or within arm 38, for example, to power and/or communicate with camera 34 and/or illumination device(s) 35. Furthermore, arm 38 may be coupled to and/or include a coupling portion 56. Coupling portion 56 may help to couple actuation device 30 (FIG. 1) to arm 38, and thus to support 36, camera 34 (FIGS. 2A, 2B, and 5A-5C), and illumination device(s) 35 (FIGS. 2A, 2B, and 5A-5C).

FIG. 3C illustrates support 36, arm 38, and coupling portion 56 separated from body portion 14C. Camera 34 (not shown in FIG. 3C), support 36, arm 38, and coupling portion 56 may form a modular camera assembly 58. Modular camera assembly 58 may also include illumination device 35 and/or one or more connection wires 54. Modular camera assembly 58 may be separable from body portion 14C of end cap 14, for example, during the cleaning of end cap 14. In this aspect, modular camera assembly 58 may be separated from body portion 14C during cleaning, for example, to clean the interior components or portions of end cap 14. Then, modular camera assembly 58 may be coupled to body portion 14C. Additionally or alternatively, body portion 14C and/or cover portion 14D may be disposable, for example, formed of plastic and removed and replaced after a procedure. In these aspects, body portion 14C and/or cover portion 14D may be more easily cleaned, or may be simply replaced with new clean components, reducing the risk of contamination between procedures.

Support 36 may include and/or may be coupled to one or more extensions 60, for example, extending radially outward from an exterior circumferential surface of support 36. Extensions 60 may be positioned within indentations (not shown) within a distal portion of body portion 14C in order to pivotably secure support 36 relative to the distal portion of body portion 14C. In one aspect, support 36 may include two extensions 60, for example, extending from opposing sides of support 36. In another aspect, support 36 may include one extension 60.

Modular camera assembly 58 may also include a spring assembly 62, for example, within interface portion 50. Spring assembly 62 may be bellows-like and may include one or more bent portions 64, for example, three bent portions 64, to bias spring assembly 62, and thus other components of modular camera assembly 58, toward a distally extended configuration. In this aspect, bent portions 64 of spring assembly 62 may bias arm 38 toward a distally extended configuration. Additionally, modular camera assembly 58 may include a base portion 66, for example, coupled to a proximal portion of spring assembly 62, and thus be a part of the modular camera assembly. For example, base portion 66 may be an extension of spring assembly 62, and base portion 66 may be formed of the same material as spring assembly 62. Alternatively, base portion 66 may be a part of body portion 14C of end cap 14, and may simply abut a proximal end of spring assembly 62 when modular camera assembly 58 is coupled to body portion 14C.

Modular camera assembly 58 may also include an indentation 68 defining an empty space. For example, indentation 68 may be adjacent to bent portions 64, such that positioning modular camera assembly 58 within body portion 14C forms a passage 70 (FIG. 3B). Furthermore, modular camera assembly 58 may include a cavity 72, for example, between bent portions 64 and arm 38. In one aspect, cavity 72 may receive a tab 74 (FIG. 4A). As discussed below, tab 74 may be movable, for example, via movement of actuation device 30, and thus control the actuation of modular camera assembly 58, for example, by proximally retracting arm 38 and/or pivoting support 36 and camera 34.

FIGS. 4A-4D illustrate details of the coupling of distal end 20A and end cap 14. FIG. 4A illustrates end cap 14 separated from distal end 20A. It is noted that, for clarity, FIG. 4A illustrates end cap 14 with cover portion 14D removed. In these aspects, end cap 14 may be coupled to distal end 20A with cover portion 14D coupled to body portion 14C of end cap 14. Alternatively, end cap 14 may be coupled to distal end 20A with cover portion 14D removed, and then cover portion 14D may be coupled to body portion 14C after end cap 14 has been coupled to distal end 20A.

As shown in FIG. 4A, end cap 14 may include slot 48 to receive a peg 46 (not shown in FIG. 4A) on distal end 20A to help couple end cap 14 to distal end 20A. As mentioned above, distal end 20A includes flap 42, and flap 42 may include opening 42A. In at least one aspect, control wire 52 may extend through shaft 20 and be secured to and/or within opening 42A. For example, control wire 52 may be coupled to second control device 28, such that movement of second control device 28 controls the movement of control wire 52 and thus the movement of flap 42. For example, movement of second control device 28 may proximally retract control wire 52 and transition flap 42 from an unactuated position, for example, substantially parallel to distal end 20A, as shown in FIG. 4A, to an actuated position. In the actuated position, flap 42 may be angled relative to distal end 20A, for example, to direct a medical device delivered through the working channel and out of distal opening 22. In one aspect, movement of second control device 28 may incrementally control the position of flap 42, for example, between the unactuated position and a plurality of actuated positions.

Moreover, distal end 20A includes tab 74. Tab 74 may include a thick portion 74A (e.g., radially thicker; protruding radially outward) and a narrow portion 74B (e.g., radially narrower). Thick portion 74A may be received within cavity 72. For example, thick portion 74A, peg 46, cavity 72, and slot 48 may be positioned on end cap 14 and distal end 20A such that positioning peg 46 in longitudinal portion 48A of slot 48 also positions thick portion 74A in passage 70. In this aspect, longitudinal portion 48A and passage 70 may be spaced apart by approximately 180 degrees around a circumference of end cap 14. Then, advancing and rotating one or more of end cap 14 and distal end 20A may advance the peg through longitudinal portion 48A and into partially circumferential portion 48B. Similarly, the advancing and rotating one or more end cap 14 and distal end 20A may also position thick portion 74A of tab 74 through passage 70 and into cavity 72, for example, aligning thick portion 74A with bent portions 64 of spring assembly 62. In this aspect, partially circumferential portion 48B may extend clockwise from longitudinal portion (i.e., when viewed from the proximal end of end cap 14), and cavity 72 may also extend clockwise from passage 70 (i.e., when viewed from the proximal end of end cap 14).

Tab 74 may also include a drive portion 74C, for example, movably and/or pivotably positioned within shaft 20. Drive portion 74C may be coupled to an actuation wire 76. Actuation wire 76 may be physically coupled to actuation device 30, such that movement of actuation device 30 controls the movement of tab 74. For example, proximally retracting actuation device 30 may proximally retract actuation wire 76, and thus proximally retract tab 74. As discussed below, proximally retracting tab 74 may proximally retract arm 38 and pivot support 36 to redirect camera 34 and/or illumination device 35 (FIGS. 2A and 2B). Furthermore, spring assembly 62 may bias tab 74 distally, for example, when the proximal force on actuation device 30 is removed. In this aspect, spring assembly 62 may then transition tab 74, actuation wire 76, and actuation device 30 to the respective original positions. Moreover, actuation wire 76 may include (e.g., enclose) one or more electrical and/or communication wires, for example, to electrically and/or communicably connect camera 34 and/or illumination device 35 (FIGS. 2A and 2B) to handle 18, for example, via connection wire(s) 54, actuation wire 76, and, as discussed below, a connection between tab 74 and modular camera assembly 58.

Distal end 20A may also include a stop surface 78, for example, positioned radially above narrow portion 74B, as shown in FIG. 4A. Stop surface 78 may limit the proximal retraction of tab 74, for example, by limiting the proximal retraction of thick portion 74A. Moreover, modular camera assembly 58 may include a mating portion 80, for example, formed by a radially inward extension forming a proximal end of cavity 72. Furthermore, although not shown, shaft 20 may include one or more lumens, for example, to movably receive control wire 52 and actuation wire 76.

FIG. 4B is an enlarged perspective view of a portion of modular camera assembly 58 of end cap 14, for example, viewing the portion of modular camera assembly 58 from a proximal position. FIG. 4B shows modular camera assembly 58 including one bent portion 64 of spring assembly 62 and mating portion 80. As shown, cavity 72 is positioned between spring assembly 62 and mating portion 80. Additionally, mating portion 80 includes one or more mating elements or pins 82. Pins 82 may help to form an electrical connection between distal end 20A and end cap 14, for example, to convey electricity (e.g. current), image signals, illumination signals, etc. between distal end 20A and end cap 14, for example, to and from camera 34 and/or illumination device(s) 35. For example, pins 82 may be electrically connected to connection wire(s) 54. Furthermore, in one or more aspects, pins 82 may be spring loaded pins, such that pins 82 may be pushed inward (i.e., distally), for example, when being coupled to tab 74 and then be biased outward (i.e., proximally) to help form the connections. Although FIG. 4B shows three pins, it is noted that mating portion 80 may include one, two, four, or more pins 82.

FIG. 4C is an enlarged perspective view of a portion (i.e., projection 44) of distal end 20A, for example, viewing the portion of distal end 20A from a distal position. FIG. 4C shows distal end 20A including tab 74, including thick portion 74A and narrow portion 74B. As shown, tab 74 includes one or more mating elements or plates 84. Each of plates 84 may be configured to mate and/or contact with one of pins 82, for example, to help form an electrical connection between distal end 20A and end cap 14, for example, to convey electricity (e.g., current), image signals, illumination signals, etc. between distal end 20A and end cap 14, for example, to and from camera 34 and/or illumination device 35. For example, plates 84 may be electrically connected to actuation wire 76. Furthermore, in one or more aspects, plates 84 may be electrical contact plates. Tab 74 may contact and push one or more of pins 82 inward (i.e., distally), as discussed above, while tab 74 is positioned within cavity 72. Then, pins 82 may be biased outward (i.e., proximally) to mate with or otherwise contact plates 84 to help form the connections. Although FIG. 4C shows three plates 84, it is noted that tab 74 may include one, two, four, or more plates 84, for example, a number and/or position to correspond to the number and/or position of pins 82.

FIG. 4D illustrates distal end 20A of shaft 20 coupled to end cap 14, forming coupling 16. It is noted that FIG. 4D illustrates end cap 14 without cover portion 14D, in order to show the internal components and/or connections. As shown, tab 74 is positioned within cavity 72 of modular camera assembly 58. Accordingly, tab 74 is adjacent to spring assembly 62. As mentioned, spring assembly 62 may bias tab 74, for example, distally. In this aspect, spring assembly 62 may help to bias tab 74 toward mating portion 80, for example, to help maintain a connection between pins 82 and plates 84 (FIGS. 4B and 4C).

Additionally, FIG. 4D includes arrow A, indicating the proximal movement of tab 74 when actuation device 30 (FIG. 1) is manipulated, for example, retracted proximally. As discussed in detail with respect to FIGS. 5A-5F, manipulation of actuation device 30, and thus movement of tab 74, proximally retracts spring assembly 62 and applies a proximal force on arm 38. Movement of arm 38 proximally may pivot support 36, and thus pivot camera 34 and illumination device(s) 35 (FIGS. 2A and 2B), for example, to change, adjust, or otherwise manipulate a field of view of camera 34 and/or a field of illumination of illumination device(s) 35.

FIGS. 5A-5F illustrate various aspects of distal end 20A and/or end cap 14, including the manipulation of support 36, and thus the manipulation of camera 34 and any illumination device(s) 35 positioned on or coupled to support 36. FIG. 5A is an end view of end cap 14 in a first configuration. FIG. 5B is a side view of end cap 14 and portions of distal end 20A (i.e., flap 42) in the first configuration, and FIG. 5C is a cross-sectional schematic view of end cap 14 in the first configuration. FIG. 5D is an end view of end cap 14 in a second configuration. FIG. 5E is a side view of end cap 14 and portions of distal end 20A (i.e., flap 42) in the second configuration, and FIG. 5F is a cross-sectional schematic view of end cap 14 in the second configuration.

As shown in FIGS. 5A-5C, camera 34 and illumination device(s) 35 are positioned on or coupled to support 36. Support 36 is coupled to arm 38. Arm 38 is biased distally by spring assembly 62, which may be at least partially enclosed between body portion 14C and cover portion 14D. Tab 74 may abut spring assembly 62 and arm 38. Arm 38 is movable relative to body portion 14C, for example, pivotable via extension(s) 60.

In the first configuration, camera 34 may have a first field of view 100, for example, substantially laterally facing relative to, and/or perpendicular to a longitudinal axis L of, end cap 14. Although not shown, illumination device(s) 35 may have a similar field of illumination as first field of view 100. In the first configuration, camera 34 may substantially face toward distal opening 22 of the working channel, for example, toward distal opening 22. In these aspects, first field of view 100 may provide a substantially side view, for example, when treating tissue to the side and/or adjacent to end cap 14. For example, first field of view 100 may be used when medical device 12 is used like a duodenoscope for an endoscopic retrograde cholangiopancreatography procedure (or ERCP). First field of view 100 also may be used when medical device is used like an endoscope in parts of the GI tract, when it is desirable to view a portion of a lumen wall to the side of medical device 12. In one aspect, first field of view 100 may be used when flap 42 is in an actuated position.

FIGS. 5D-5F show end cap 14 in the second configuration. In the second configuration, camera 34 is pivoted to have a second field of view 200, for example, substantially distal facing. Although not shown, illumination device(s) 35 may have a similar field of illumination as second field of view 200. As shown in FIG. 5F, arm 38 is retracted proximally in order to transition to the second configuration. As discussed above, manipulation of actuation device 30 (i.e., proximal retraction within actuation device slot 30A) may retract tab 74 and arm 38, for example, via the interaction of actuation wire 76, tab 74, and cavity 72 (FIGS. 4A-4D). Retracting arm 38 contracts spring assembly 62, and spring assembly 62 biases arm 38 distally once the retracting force is reduced or removed. Furthermore, retracting arm 38 pivots support 36 relative to body portion 14C, for example, via extension(s) 60, and thus pivots the field of view of camera 34, along with the field of illumination of any illumination device(s) 35.

In these aspects, second field of view 200 is angled distally toward the longitudinal axis L of end cap 14 compared to first field of view 100. For example, relative to first field of view 100, second field of view 200 may be angled from 1 degree to approximately 90 degrees (i.e., distally facing; parallel to axis L), or more than 90 degrees to provide more view to the side of device 12 opposite that seen by field of view 100. For example, second field of view 200 may be angled approximately 45 degrees relative to first field of view 100 and the longitudinal axis L. In another aspect, second field of view 200 may be angled approximately 80 degrees relative to first field of view, such that second field of view 200 is angled approximately 10 degrees from the longitudinal axis L of end cap 14. In these aspects, second field of view 200 may provide a substantially front view, for example, when treating tissue distal to end cap 14. For example, second field of view 200 may be used when medical device 12 is used like a duodenoscope for assisting in navigating to the desired location. Second field of view 200 also may be used when medical device 12 is used like an endoscope, which typically has a front-facing camera. In one aspect, second field of view 200 may be used when flap 42 is in an unactuated position.

Moreover, in one or more aspects, the position of arm 38, and thus the positions of support 36 and camera 34, may be adjustable in order to provide an adjustable field of view. For example, a user may manipulate actuation device 30 to adjust the field of view of camera 34 in order for camera 34 to capture images of (or for the one or more illumination devices 35 on support 36 to illuminate) one or more regions of the treatment site adjacent to and/or distal of distal end 20A. For example, support 36 may be angled such that camera 34 captures the one or more treatment devices that are delivered through the working channel and out of distal opening 22 and directed by flap 42. As mentioned above, support 36 may be pivotable through a plurality of positions, such that the field of view of camera 34 and the field of illumination of illumination device(s) 35 is adjustable through a plurality of angles.

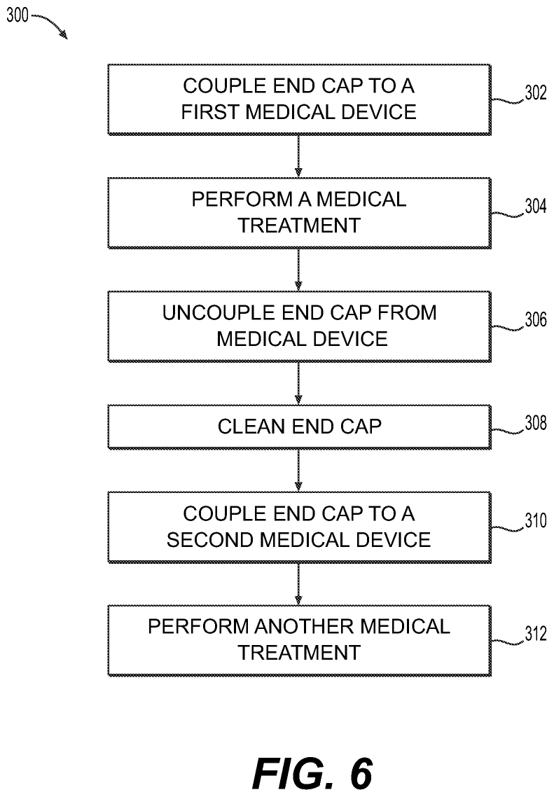

FIG. 6 illustrates a method 300 that may be performed with end cap 14. In these aspects, end cap 14 (or portions of end cap 14) may be reusable, and medical device 12 may be disposable. Step 302 includes coupling end cap 14 to medical device 12, for example, coupling end cap 14 to distal end 20A of shaft 20 of medical device 12 to form coupling 16. As discussed above, coupling end cap 14 to medical device 12 may include aligning peg 46 with slot 48, and aligning tab 74 with passage 70. Furthermore, coupling end cap 14 to medical device 12 may include manipulating end cap 14 and/or medical device 12 such that peg 46 is positioned within partially circumferential portion 48B, and such that tab 74 is positioned within cavity 72. Positioning peg 46 within partially circumferential portion 48B and/or positioning tab 74 within cavity 72 may form a snap-lock, friction fit, or other appropriate coupling. In this aspect, positioning tab 74 within cavity 72 may also electrically connect medical device 12 and end cap 14, for example, via pin(s) 82 and plate(s) 84.

Next, a step 304 includes performing a medical treatment at the treatment site. For example, step 304 may include inserting a treatment device through the working channel of medical device 12, for example, through port 24 and out of distal opening 22. Step 304 may include deflecting distal end 20A of medical device 12, for example, via one or more of knobs 26A, 26B of first control device 26. Additionally, step 304 may include directing the treatment device inserted through medical device 12, for example, by directing the treatment device with flap 42. In this aspect, flap 42 may be manipulated via connection wire 52, which may be controlled via second control device 28. Furthermore, step 304 may include directing camera 34 and/or illumination device 35. For example, directing camera 34 and/or illumination device 35 may include proximally retracting tab 74, for example, via actuation device 30 and actuation wire 76. As discussed above, proximally retracting tab 74 may retract arm 38, for example, to pivot support 36 and direct camera 34 and/or illumination device 35.

A step 306 includes uncoupling end cap 14 from medical device 12, after removing medical device 12 and end cap 14 from a body of a patient. For example, one or more of end cap 14 and/or distal end 20A of shaft 20 of medical device 12 may be rotated and/or manipulated. In this aspect, peg 46 may be removed from slot 48, and tab 74 may be removed from cavity 72 and passage 70.

Next, a step 308 includes cleaning end cap 14. For example, end cap 14 may be immersed in a cleaning or sterilizing solution. In one or more aspects, cleaning end cap 14 may include separating components of end cap 14. For example, body portion 14C, cover portion 14D, and modular camera assembly 58 may be separated. Each component of end cap 14 may be cleaned, for example, immersed in a cleaning solution. Separating components of end cap 14 may help to allow the cleaning solution to contact the entire surface area of each component of end cap 14. Alternatively or additionally, one or more components of end cap 14 may be disposable. For example, body portion 14C and/or cover portion 14D may be formed of a low-cost material (e.g., a plastic) and/or may be disposable. In this aspect, modular camera assembly 58 may be cleaned, and then be coupled to a new body portion 14C and/or a new cover portion 14D.

Then, a step 310 includes coupling end cap 14 to another or second medical device 12. Step 310 may include one or more of the steps discussed above with respect to step 302. In this aspect, a distal end 20A of shaft 20 of the second medical device 12 may be aligned with and coupled to end cap 14 to form coupling 16, as discussed above.

A step 312 includes performing another medical treatment, for example, at a different treatment site. Step 312 may include one or more of the steps discussed above with respect to 304. Additionally, the steps of method 300 may be repeated as many times as necessary, for example, reusing end cap 14 or portions of end cap 14 (e.g., modular camera assembly 58) as many times as necessary. Reusing end cap 14 or portions of end cap 14 may help to reduce costs. Additionally, coupling end cap 14 to a different medical device 12 may help to reduce the risk of contamination between different treatment sites. Furthermore, end cap 14 and/or portions of end cap 14 may be unassembled, which may help clean end cap 14 and/or portions of end cap 14. Alternatively or additionally, one or more components of end cap 14 may be replaced. For example, camera 34 and/or illumination device 35 may be replaced, upgraded, etc.

While principles of this disclosure are described herein with reference to illustrative aspects for various applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

We claim:

1. A medical system, comprising:
a medical device, including:
a handle, including an actuation device; and
a shaft extending from the handle, wherein a distal end of the shaft includes a tab, and wherein the tab is movable based on movement of the actuation device; and
an end cap, including:
a modular camera assembly, wherein the modular camera assembly includes a camera, a support, an arm, and a cavity,
wherein the tab is coupleable to the modular camera assembly via the cavity, and wherein movement of the tab controls a position of the support and the camera.

2. The medical system of claim 1, wherein the actuation device is physically and electrically connected to the tab via an actuation wire.

3. The medical system of claim 2, wherein the modular camera assembly includes one or more mating elements, wherein the tab includes one or more mating elements on a distal end of the tab, and wherein the one or more mating elements on the modular camera assembly and the one or more mating elements on the tab are configured to electrically connect the handle to the camera.

4. The medical system of claim 3, wherein the arm further includes one or more connection wires that electrically connect the camera to the one or more mating elements on the modular camera assembly.

5. The medical system of claim 1, wherein the distal end of the shaft includes a peg, and wherein the end cap includes a slot configured to receive the peg.

6. The medical system of claim 5, wherein the slot includes a longitudinal portion and a partially circumferential portion extending from the longitudinal portion.

7. The medical system of claim 6, wherein the modular camera assembly and a body portion of the end cap form a passage that connects to the cavity, and wherein the longitudinal portion of the slot is spaced away from the passage along an interior circumference of the end cap.

8. The medical system of claim 1, wherein the handle includes a control device, wherein the distal end of the shaft includes a flap, and wherein movement of the control device is configured to adjust a position of the flap between at least a first configuration and a second configuration.

9. The medical system of claim 8, wherein, when the end cap is coupled to the distal end of the shaft, the flap at least partially aligns with the modular camera assembly.

10. The medical system of claim 1, wherein the modular camera assembly includes a spring assembly configured to bias the tab distally.

11. The medical system of claim 1, wherein the end cap further includes a body portion and a cover portion, and wherein the support is pivotably coupled the body portion of the end cap.

12. The medical system of claim 11, wherein the body portion, the cover portion, and the modular camera assembly are separable and coupleable.

13. The medical system of claim 1, wherein the handle includes a port, and wherein the port is in communication with a distal opening in the distal end of the shaft.

14. The medical system of claim 13, wherein the end cap includes a proximal portion and a distal portion, wherein the proximal portion is substantially cylindrical, and wherein the distal portion is substantially semi-cylindrical.

15. The medical system of claim 1, wherein the end cap further includes one or more illumination devices positioned on the support.

16. An end cap for a medical device, comprising:
a body portion;
a cover portion, and
a modular camera assembly, wherein the modular camera assembly includes a camera, a support, an arm, and a cavity, wherein the camera is coupled to the support, and wherein movement of the arm is configured to rotate the support and adjust a field of view of the camera.

17. The end cap of claim 16, wherein the modular camera assembly further includes one or more mating elements, and wherein the arm further includes one or more connection wires that electrically connect the camera to the one or more mating elements.

18. The end cap of claim 17, further comprising an illumination device coupled to the support, wherein the one or more connection wires electrically connect the illumination device to the one or more mating elements.

19. A method, comprising:
coupling an end cap to a distal end of a shaft of a first medical device;
wherein the end cap includes a modular camera assembly, wherein the modular camera assembly includes a camera, a support, an arm, and a cavity, and
wherein the first medical device includes a handle and a shaft, wherein the handle includes an actuation device, wherein a distal end of the shaft includes a tab, wherein the tab is physically coupled to the actuation device via an actuation wire, and wherein the end cap and the shaft are coupled by at least positioning the tab within the cavity;
performing a first medical treatment, wherein performing the medical treatment includes either directing a treatment device with an elevator of the first medical device or directing a field of view of the camera;
uncoupling the end cap from the distal end of the shaft of the first medical device;
cleaning the end cap;
coupling the end cap to a second medical device; and
performing a second medical treatment.

20. The method of claim 19, further comprising:
wherein the end cap further includes a body portion and a cover portion, and wherein the step of cleaning the end cap includes separating the body portion, the cover portion, and the modular camera assembly.
